(19) United States
(12) Patent Application Publication
(10) Pub. No.: US 2025/0235568 A1
(43) Pub. Date: Jul. 24, 2025

(54) ATOMIZER AND ATOMIZING METHOD

(71) Applicants: NALUX CO., LTD., Osaka (JP); TOHOKU UNIVERSITY, Sendai-shi (JP)

(72) Inventors: Shota SASAKI, Sendai-shi (JP); Toshiro KANEKO, Sendai-shi (JP); Seitaro KITAGAWA, Osaka (JP); Takashi MARUKO, Osaka (JP)

(73) Assignees: NALUX CO., LTD., Osaka (JP); TOHOKU UNIVERSITY, Sendai-shi (JP)

(21) Appl. No.: 19/175,406

(22) Filed: Apr. 10, 2025

Related U.S. Application Data
(63) Continuation of application No. PCT/JP2022/041159, filed on Nov. 4, 2022.

(51) Int. Cl. A61L 2/14; A61L 2/22

(52) U.S. Cl. CPC A61L 2/14; A61L 2/22; A61L 2202/11; A61L 2202/15

(57) ABSTRACT

An atomizing method comprises providing an atomizer including a mist generator and a plasma producer. The plasma producer includes a tube of dielectric material, an external electrode on an outside surface of the tube of dielectric material, an internal electrode on an inside surface of the tube of dielectric material, and a high-frequency power supply for applying a high-frequency voltage between the external electrode and the internal electrode. Mist from the mist generator is plasma-activated while passing through the tube of dielectric material and emitted from an end of the tube of dielectric material. The external electrode and the internal electrode are located such that location of the external electrode and location of the internal electrode does not overlap or merely partially overlap in the longitudinal direction of the tube of dielectric material. Peroxynitrous acid is continuously generated by generating hydrogen peroxide and nitrous acid using plasma-activated water mist.

### CROSS REFERENCE TO RELATED APPLICATION

This is a Continuation of International Patent Application No. PCT/JP2022/041159 filed Nov. 4, 2022, which designates the U.S. The content of this application is hereby incorporated by reference.

### TECHNICAL FIELD

The subject disclosure relates to an atomizer and an atomizing method.

### BACKGROUND ART

An atomizer and an atomizing method in which peroxynitrous acid (HOONO) having sterilizing effect is generated by plasma-activating mist generated from liquid consisting mainly of water have been developed (Patent document 1). FIG. 7 of Patent document 1 shows a plasma producer of an atomizer, which produces plasma-activated mist. The above-described plasma producer is configured such that by applying a high-frequency voltage between an internal electrode installed inside a tube and an external electrode installed outside the tube, mist which contains micro droplets and air has been fed into the tube and is plasma-activated to generate peroxynitrous acid (HOONO).

A generation speed of peroxynitrous acid (HOONO) of the above-described plasma producer is not sufficiently high.

Accordingly, there is a need for an atomizer and an atomizing method by which peroxynitrous acid (HOONO) can be generated at a sufficiently high generation speed.

### PATENT DOCUMENT

Patent document 1: WO2014/145570

### SUMMARY

An atomizing method in which peroxynitrous acid is continuously generated by generating hydrogen peroxide and nitrous acid using plasma-activated water mist, the method using an atomizer provided with a mist generator and a plasma producer, wherein the plasma producer is provided with a tube of dielectric material, an external electrode installed on an outside surface of the tube of dielectric material, an internal electrode installed on an inside surface of the tube of dielectric material and a high-frequency power supply for applying a high frequency voltage between the external electrode and the internal electrode and is configured such that mist that is sent from the mist generator to the tube of dielectric material is plasma-activated while passing through the tube of dielectric material and emitted from an end of the tube of dielectric material and wherein the external electrode and the internal electrode are located such that location of the external electrode and location of the internal electrode does not overlap or merely partially overlap in the longitudinal direction of the tube of dielectric material.

In other words, embodiments of the invention include an atomizing method, comprising providing an atomizer including a mist generator and a plasma producer. The plasma producer is provided with a tube of dielectric material, an external electrode installed on an outside surface of the tube of dielectric material, an internal electrode installed on an inside surface of the tube of dielectric material and a high-frequency power supply for applying a high-frequency voltage between the external electrode and the internal electrode. Mist that is sent from the mist generator to the tube of dielectric material is plasma-activated while passing through the tube of dielectric material and emitted from an end of the tube of dielectric material. The external electrode and the internal electrode are located such that location of the external electrode and location of the internal electrode does not overlap or merely partially overlap in the longitudinal direction of the tube of dielectric material. Peroxynitrous acid is continuously generated by generating hydrogen peroxide and nitrous acid using plasma-activated water mist, using the atomizer.

### BRIEF DESCRIPTION OF DRAWINGS

FIG. 1 shows an atomizer according to an embodiment;

FIG. 2 shows an example of the structure of the plasma producer according to the present embodiment;

FIG. 3 shows an example of the structure of a conventional plasma producer;

FIG. 4 shows an amount of mist emitted by the plasma generator of the structure of surface DBD shown in FIG. 2 and that emitted by the plasma generator of the structure of volume DBD shown in FIG. 3;

FIG. 5 shows the structure of a plasma producer of the structure of surface DBD used for the measurement of the generation rate of peroxynitrous acid (HOONO);

FIG. 6 shows a generation rate of peroxynitrous acid (HOONO) of a plasma generator of the structure of surface DBD and a generation rate of peroxynitrous acid (HOONO) of a plasma generator of the structure of volume DBD;

FIG. 7 shows concentration of hydrogen peroxide, nitrous acid, nitrate ion and nitrite ion generated by the plasma generator of the structure of surface DBD;

FIG. 8 shows concentration of hydrogen peroxide, nitrous acid, nitrate ion and nitrite ion generated by the plasma generator of the structure of volume DBD;

FIG. 9 shows concentration of nitrous acid shown in FIG. 7 and that shown in FIG. 8, the scale on the vertical axis of FIG. 9 covering a smaller range than in FIG. 7 and FIG. 8;

FIG. 10 shows concentration of hydrogen peroxide, nitrous acid, nitrate ion and nitrite ion generated by the plasma generator of the structure of surface DBD when water solution of sodium nitrite is used;

FIG. 11 shows generation rate of peroxynitrous acid (HOONO) of the plasma generator of the structure of surface DBD when water solution of sodium nitrite is used;

FIG. 12 shows a change in concentration of the species obtained by the simulation when concentration of sodium nitrite in the water solution is 1200 micro-molar;

FIG. 13 shows a change in concentration of the species obtained by the simulation when concentration of sodium nitrite in the water solution is 2000 micro-molar; and

FIG. 14 shows a change in concentration of peroxynitrous acid (HOONO) obtained by the simulation when concentration of sodium nitrite in the water solution is 1200 micro-molar and a change in concentration of peroxynitrous acid (HOONO) obtained by the simulation when concentration of sodium nitrite in the water solution is 2000 micro-molar.

### DESCRIPTION OF EMBODIMENTS

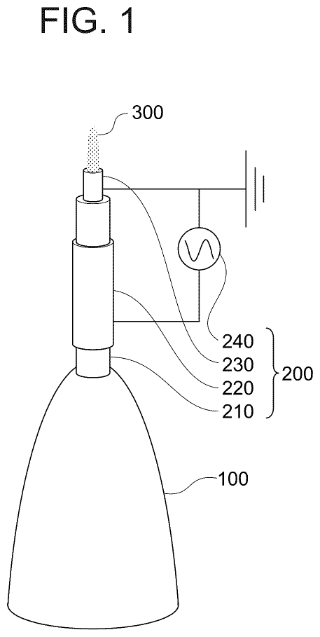

FIG. 1 shows an atomizer according to an embodiment. The atomizer that produces plasma-activated mist includes a mist generator 100 and a plasma producer 200. The mist generator 100 can be a one that generates mist by an ultrasonic vibrator from liquid consisting mainly of water in a tank. The ultrasonic vibrator is the ultrasonic nebulizer NB-80E-01-H of TDK, for example. A diameter of droplets of mist is in a range from 0.5 micrometers to 30 micrometers. The plasma producer 200 is provided with a tube 210 made of a dielectric, an external electrode 220 installed outside the tube 210, an internal electrode 230 installed inside the tube 210 and a high-frequency power source 240. Each of the external electrode 220 and the internal electrode 230 is also in the shape of a tube. The internal electrode 230 in the shape of a tube protrudes above the top end of the tube 210. The high-frequency power source 240 is connected to the external electrode 220 and the internal electrode 230 such that a high frequency voltage can be applied therebetween. A terminal of the high-frequency power source 240 connected to the internal electrode 230 is grounded. In general, a frequency of the high-frequency power source 240 is in a range from 1 kilohertz to 100 kilohertz. The frequency is more preferably in a range from 5 kilohertz to 30 kilohertz. A peak-to-peak value of voltage of the high frequency power source 240 is in a range from 1 kilovolt to 30 kilovolts.

Mist generated by the mist generator 100 is sent from the bottom end of the tube 210 shown in FIG. 1 to the plasma producer 200. A flow rate of the mist is preferably in a range from 0.01 micro-litters per second (μL/s) to 1000 micro-litters per second (μL/s). When a high-frequency voltage is applied between the external electrode 220 and the internal electrode 230 by the high-frequency power source 240, the mist is plasma-activated and the plasma-activated mist is emitted from the top end of the internal electrode 230 that is in the shape of a tube and is protruding above the top end of the tube 210.

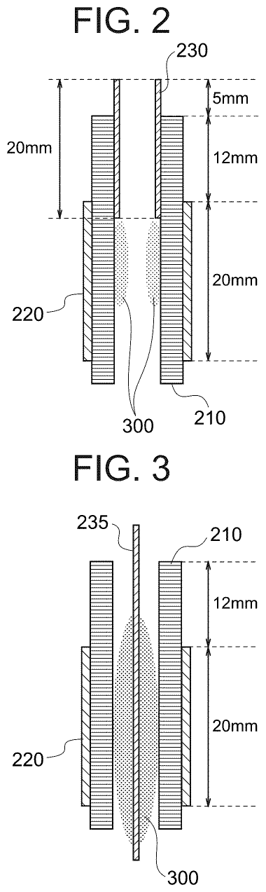

FIG. 2 shows an example of the structure of the plasma producer 200 according to the present embodiment. A length in the longitudinal direction of the tube 210 is 34 millimeters. A diameter of the tube 210 is 3.9 millimeters. A wall thickness of the tube 210 is 0.7 millimeters. In general, a diameter of the tube made of dielectric is preferably in a range from 2 millimeters to 10 millimeters. In the example, material of the tube 210 is quartz glass and the external electrode 220 installed outside the tube 210 is a conductive and adhesive tape that covers the outer surface of the tube 210. The tape is made of copper-foil. A width (a length in the longitudinal direction of the tube 210) of the copper-foil tape is 20 millimeters and a thickness of the copper-foil tape is 0.7 millimeters. The top end of the external electrode 220 is located 12 millimeters below the top end of the tube 210. In the example, the internal electrode 230 installed inside the tube 210 is a tube made of stainless steel. A length in the longitudinal direction of the tube made of stainless steel is 20 millimeters. An outside diameter of the tube made of stainless steel is 2.41 millimeters and a wall thickness of the tube made of stainless steel is 0.21 millimeters. A length in the longitudinal direction of an overlapping area of the external electrode 220 and the internal electrode 230 is 3 millimeters. The internal electrode 230 is protruding above the top end of the tube 210 and the length in the longitudinal direction of the internal electrode 230 above the top end of the tube 210 is 5 millimeters. In the present embodiment, the plasma-activated mist is formed on an area of the inner surface of the tube 210, the area being below and adjacent to the bottom end of the internal electrode 230 and surrounded by the external electrode 220.

In general, a position of the external electrode and a position of the internal electrode in in the longitudinal direction of the tube 210 are made different from each other. In order that electrical discharge between both of the electrodes can easily occur, it is preferable to locate both of the electrodes such that they partially overlap with each other in in the longitudinal direction of the tube 210.

FIG. 3 shows an example of the structure of a conventional plasma producer. Sizes and material of a tube 210 are identical with those of the example described above and sizes and material of an external electrode 220 installed outside the tube 210 are identical with those of the example described above. The top end of the external electrode 220 is located 12 millimeters below the top end of the tube 210. An internal electrode 235 is in the shape of a rod. A length of the rod is 100 millimeters and a diameter of the rod is 0.9 millimeters. Material of the internal electrode 235 is tungsten. The internal electrode 235 is located at the position of the central axis of the tube 210. In the conventional plasma producer, plasma-activated mist is formed in a space inside the tube 210 in which a position in the longitudinal direction of the external electrode 220 and that of the internal electrode 230 overlaps each other.

The plasma generators shown in FIG. 2 and FIG. 3 generate plasma through dielectric barrier discharge (DBD). In the present specification, the structure of the plasma generator 200 shown in FIG. 2 is referred to as a structure of surface dielectric barrier discharge (a structure of surface DBD) and the structure of the plasma generator shown in FIG. 3 is referred to as a structure of volume dielectric barrier discharge (a structure of volume DBD).

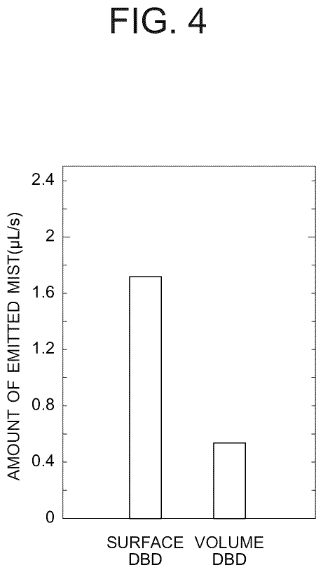

FIG. 4 shows an amount of mist emitted by the plasma generator of the structure of surface DBD shown in FIG. 2 and that emitted by the plasma generator of the structure of volume DBD shown in FIG. 3. A frequency of the high-frequency power source 240 is 9 kilohertz and a peak-to-peak value of voltage is 18 kilovolts. In order to measure an amount of emitted mist, mist emitted by the plasma generator was made to adhere to a glass plate. An amount of mist emitted by the plasma generator of the structure of surface DBD is approximately 1.7 micro-litters per second (μL/s) and an amount of mist emitted by the plasma generator of the structure of volume DBD is approximately 0.5 micro-litters per second (μL/s). The reason why the amount of mist emitted by the plasma generator of the structure of surface DBD is greater than the amount of mist emitted by the plasma generator of the structure of volume DBD is estimated as below. In the case of the plasma generator of the structure of volume DBD, a high-intensity electric field that is required to produce plasma is applied to all the area of a cross section of the tube and charged particles of mist more easily adhere to the wall surface. Accordingly, from the standpoint of an amount of emitted mist, a plasma generator of the structure of surface DBD is preferable to a conventional plasma generator of the structure of volume DBD.

Sterilizing power of plasma-activated mist will be described below. Peroxynitrous acid (HOONO) has sterilizing power and there exists a definite correlation between the sterilizing power and a generation rate of peroxynitrous acid (HOONO). Peroxynitrous acid (HOONO) can be continuously generated from hydrogen peroxide and nitrous acid and hydrogen peroxide and nitrous acid can be produced by plasma-activating microscopic droplets of water flying in the air, that is mist.

Thus, a generation rate of peroxynitrous acid (HOONO) of a plasma generator of the structure of surface DBD and that of a plasma generator of the structure of volume DBD were measured.

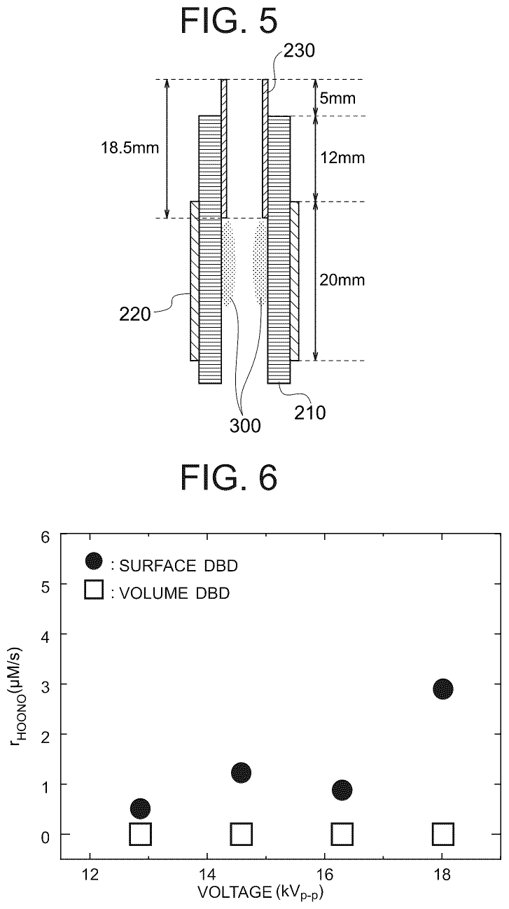

FIG. 5 shows the structure of a plasma producer of the structure of surface DBD used for the measurement of a generation rate of peroxynitrous acid (HOONO). A length in the longitudinal direction of a tube 210 is 34 millimeters. A diameter of the tube 210 is 3.9 millimeters. A wall thickness of the tube 210 is 0.7 millimeters. Material of the tube 210 is quartz glass. An external electrode 220 installed outside the tube 210 is a conductive and adhesive copper-foil tape. A width (a length in the longitudinal direction of the tube 210) of the copper-foil tape is 20 millimeters and a thickness of the copper-foil tape is 0.7 millimeters. The top end of the external electrode 220 is located 12 millimeters below the top end of the tube 210. An internal electrode 230 installed inside the tube 210 is a tube made of stainless steel. A length in the longitudinal direction of the tube made of stainless steel is 18.5 millimeters. An outside diameter of the tube made of stainless steel is 2.41 millimeters and a wall thickness of the tube made of stainless steel is 0.21 millimeters. A length in the longitudinal direction of an overlapping area of the external electrode 220 and the internal electrode 230 is 1.5 millimeters. The internal electrode 230 is protruding above the top end of the tube 210 and a length in the longitudinal direction of the internal electrode 230 above the top end of the tube 210 is 5 millimeters. Plasma-activated mist is formed on an area of the inner surface of the tube 210, the area being below and adjacent to the bottom end of the internal electrode 230 and surrounded by the external electrode 220.

A plasma producer of the structure of volume DBD used for the measurement of the generation rate is that shown in FIG. 3.

FIG. 6 shows a generation rate of peroxynitrous acid (HOONO) of the plasma generator of the structure of surface DBD and a generation rate of peroxynitrous acid (HOONO) of the plasma generator of the structure of volume DBD. The horizontal axis of FIG. 6 indicates a peak-to-peak value of voltage applied between both electrodes. The unit of voltage is kilovolt. The vertical axis of FIG. 6 indicates a generation rate of peroxynitrous acid (HOONO). The unit of a generation rate of peroxynitrous acid (HOONO) is micro-molar per second (μM/s). Molar concentration is represented by M and one molar means that a solution contains one mole of solute per litter of solvent. How to measure a generation rate of peroxynitrous acid (HOONO) will be described later.

According to FIG. 6, a generation rate of peroxynitrous acid (HOONO) of the plasma generator of the structure of surface DBD is approximately 3 micro-molar per second (μM/s) in the case of a peak-to-peak value of voltage of 18 kv while a generation rate of peroxynitrous acid (HOONO) of the plasma generator of the structure of volume DBD is zero in the case of a peak-to-peak value of voltage of 18 kv. Accordingly, from the standpoint of generation rate of peroxynitrous acid (HOONO) the plasma generator of the structure of surface DBD is preferable to the plasma generator of the structure of volume DBD.

Peroxynitrous acid (HOONO) is generated according to the following reaction formula (1).

H₂O₂+HNO₂+H⁺→HOONO+H₂O+H⁺ (1)

The species on the left side of the reaction formula (1), that is hydrogen peroxide, nitrous acid and hydrogen ion
H⁺
   are generated by plasma-activated mist and remain for a long time and generation of peroxynitrous acid (HOONO) continues. Peroxynitrous acid (HOONO) is then resolved into nitrate ion
NO₃⁻
   and nitrite ion
NO₂⁻2.
   A half-life period of peroxynitrous acid (HOONO) is typically approximately 1 second. The generation continues until hydrogen peroxide is exhausted.

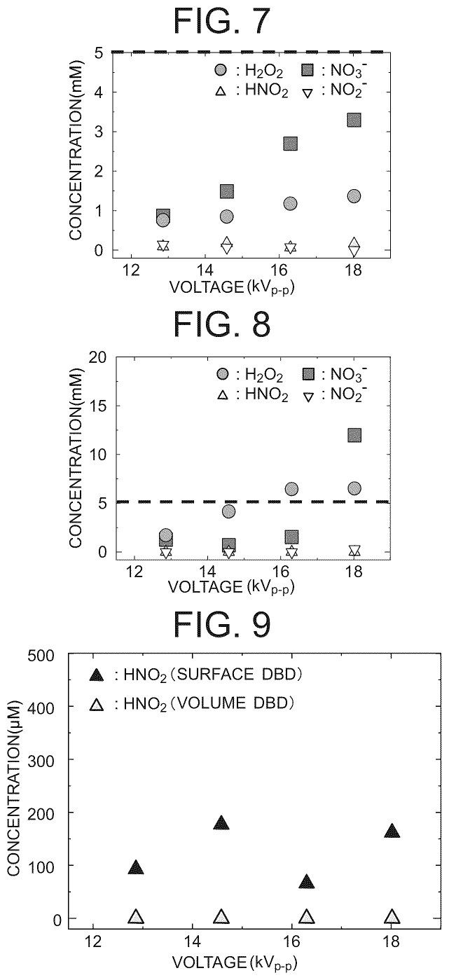

FIG. 7 shows concentration of hydrogen peroxide, nitrous acid, nitrate ion and nitrite ion generated by the plasma generator of the structure of surface DBD. The horizontal axis of FIG. 7 indicates a peak-to-peak value of voltage applied between both electrodes. The unit of voltage is kilovolts. The vertical axis of FIG. 7 indicates concentration of the species. The unit of concentration is milli-molar. The concentration of the species was measured by applying absorptiometry using ultra micro-cells to droplets gathered from mist using a glass plate or the like.

FIG. 8 shows concentration of hydrogen peroxide, nitrous acid, nitrate ion and nitrite ion generated by the plasma generator of the structure of volume DBD. The horizontal axis of FIG. 8 indicates a peak-to-peak value of voltage applied between both electrodes. The unit of voltage is kilovolts. The vertical axis of FIG. 8 indicates concentration of the species. The unit of concentration is milli-molar. The range covered by the scale on the vertical axis of FIG. 8 is larger than the range covered by the scale on the vertical axis of FIG. 7.

When FIG. 7 and FIG. 8 are observed, in FIG. 8 concentration of hydrogen peroxide is approximately 6 milli-molar in the case of a peak-to-peak value of voltage of 18 kv while in FIG. 7 concentration of hydrogen peroxide is approximately 1.3 milli-molar in the case of a peak-to-peak value of voltage of 18 kv. In other words, concentration of hydrogen peroxide in the case of the plasma generator of the structure of volume DBD shown in FIG. 8 is approximately five times as great as concentration of hydrogen peroxide in the case of the plasma generator of the structure of surface DBD shown in FIG. 7.

FIG. 9 shows concentration of nitrous acid shown in FIG. 7 and that shown in FIG. 8, the scale on the vertical axis of FIG. 9 covering a smaller range than in FIG. 7 and FIG. 8. The horizontal axis of FIG. 9 indicates a peak-to-peak value of voltage applied between both electrodes. The unit of voltage is kilovolts. The vertical axis of FIG. 9 indicates concentration of nitrous acid. The unit of concentration is micro-molar. According to FIG. 9, concentration of nitrous acid generated by the plasma generator of the structure of surface DBD is approximately 170 micro-molar in the case of a peak-to-peak value of voltage of 18 kv while concentration of nitrous acid generated by the plasma generator of the structure of volume DBD is zero in the case of a peak-to-peak value of voltage of 18 kv. “Zero” means that concentration of nitrous acid generated by the plasma generator of the structure of volume DBD is smaller than 10 micro-molar, because the limit of detection of concentration of nitrous acid is 10 micro-molar.

According to values shown in FIGS. 6 to 9, it can be estimated that in order to increase a generation rate of peroxynitrous acid (HOONO), keeping ratios between amounts of the species and hydrogen ion in the reaction formula (1) at appropriate values (ideally, each species should be of an equal amount) is important and that concentration of nitrous acid has particularly a great effect on the generation rate of peroxynitrous acid (HOONO). It is estimated that the reason why in the case of the conventional plasma generator of the structure of volume DBD, a generation rate of peroxynitrous acid (HOONO) is lower than in the case of the plasma generator of the structure of surface DBD is that in the case of the conventional plasma generator of the structure of volume DBD, concentration of hydrogen peroxide is higher than in the case of the plasma generator of the structure of surface DBD, but concentration of nitrous acid is below the limit of detection.

Based on the assumption that concentration of nitrous acid has a great effect on the generation rate of peroxynitrous acid (HOONO), the inventors tried to increase concentration of nitrous acid by using water solution of sodium nitrite in place of water to form mist in the case of the plasma generator of the structure of surface DBD.

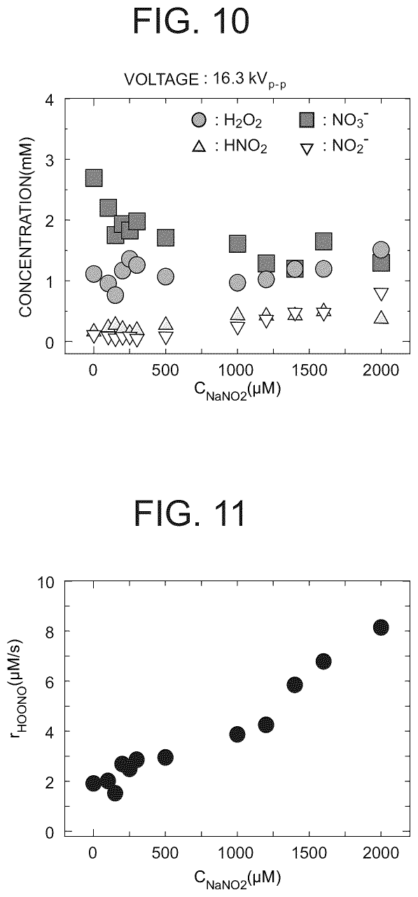

FIG. 10 shows concentration of hydrogen peroxide, nitrous acid, nitrate ion and nitrite ion generated by the plasma generator of the structure of surface DBD when water solution of sodium nitrite is used. The horizontal axis of FIG. 10 indicates concentration of sodium nitrite in the water solution. The unit of concentration is micro-molar. The vertical axis of FIG. 10 indicates concentration of species. The unit of concentration is milli-molar. A peak-to-peak value of voltage applied between both electrodes is 16.3 kilovolts.

FIG. 11 shows generation rate of peroxynitrous acid (HOONO) of the plasma generator of the structure of surface DBD when water solution of sodium nitrite is used. The horizontal axis of FIG. 11 indicates concentration of sodium nitrite in the water solution. The unit of concentration is micro-molar. The vertical axis of FIG. 11 indicates generation rate of peroxynitrous acid (HOONO). The unit of generation rate is micro-molar per second (μM/s).

How to obtain a generation rate of peroxynitrous acid (HOONO) will be described below. From the reaction formula (1), a generation rate of peroxynitrous acid (HOONO) can be expressed by the following formula (2).

r_{HOONO}=k[H₂O₂][HNO₂][H⁺]≅k[H₂O₂][HNO₂][NO₃⁻] (2)

k represents reaction rate constant and the following value is employed.

k=9.6×10³(M⁻²s⁻¹)

Values of generation rate of peroxynitrous acid (HOONO) were obtained by substituting values of concentration of the species measured by the method described above into the formula (2).

The reason why values of generation rate of peroxynitrous acid (HOONO) of the plasma generator of the structure of volume DBD shown in FIG. 6 are zero is that values of concentration of nitrous acid generated by the plasma generator of the structure of volume DBD are below the limit of detection and regarded as zero.

According to FIG. 11, the generation rate of peroxynitrous acid (HOONO) increases with concentration of sodium nitrite in the water solution. For practical applications, the concentration of sodium nitrite in the water solution should preferably be in a range from 1 micro-molar to 100 milli-molar.

A change in concentration of the species and peroxynitrous acid (HOONO) with passage of time will be estimated through simulation below. The simulation is that for zero-order chemical reaction and employs plural nonequilibrium and equilibrium reaction formulars. In the simulation a change in concentration of the species and peroxynitrous acid (HOONO) with passage of time were obtained by an implicit method using the Newton-Raphson method.

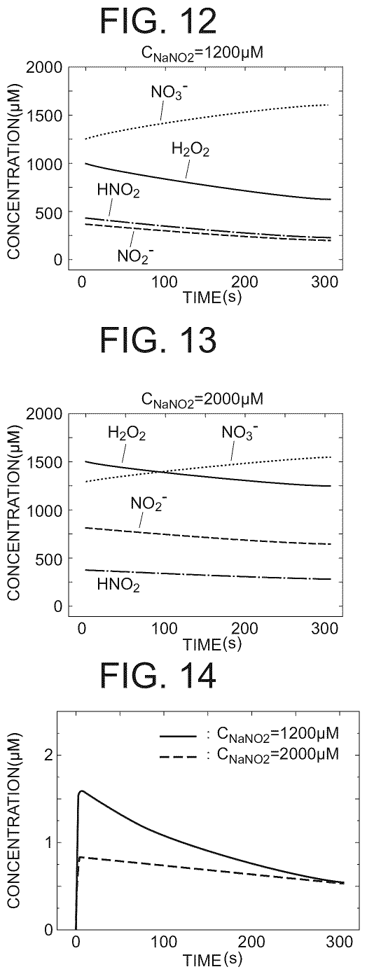

FIG. 12 shows a change in concentration of the species obtained by the simulation when concentration of sodium nitrite in the water solution is 1200 micro-molar. The horizontal axis of FIG. 12 indicates time. The unit of time is second. The vertical axis of FIG. 12 indicates concentration of the species. The unit of concentration is micro-molar. As initial values of the species, the values shown in FIG. 10 were used.

FIG. 13 shows a change in concentration of the species obtained by the simulation when concentration of sodium nitrite in the water solution is 2000 micro-molar. The horizontal axis of FIG. 13 indicates time. The unit of time is second. The vertical axis of FIG. 13 indicates concentration of the species. The unit of concentration is micro-molar. As initial values of the species, the values shown in FIG. 10 were used.

FIG. 14 shows a change in concentration of peroxynitrous acid (HOONO) obtained by the simulation when concentration of sodium nitrite in the water solution is 1200 micro-molar and a change in concentration of peroxynitrous acid (HOONO) obtained by the simulation when concentration of sodium nitrite in the water solution is 2000 micro-molar. The horizontal axis of FIG. 14 indicates time. The unit of time is second. The vertical axis of FIG. 14 indicates concentration of the species. The unit of concentration is micro-molar.

According to FIG. 14, concentration of peroxynitrous acid (HOONO) is kept at a value of 0.5 micro-molar or greater for a period of 300 seconds or more in the case of concentration of sodium nitrite in the water solution of 1200 micro-molar and in the case of concentration of sodium nitrite in the water solution of 2000 micro-molar. The initial value of concentration of peroxynitrous acid (HOONO) in the case of concentration of sodium nitrite in the water solution of 1200 micro-molar is greater than that in the case of concentration of sodium nitrite in the water solution of 2000 micro-molar. On the other hand, a rate at which concentration of peroxynitrous acid (HOONO) decreases for a certain period in the case of concentration of sodium nitrite in the water solution of 1200 micro-molar is greater than that in the case of concentration of sodium nitrite in the water solution of 2000 micro-molar. The way of a change in concentration of peroxynitrous acid (HOONO) with passage of time in the case of concentration of sodium nitrite in the water solution of 1200 micro-molar is appropriate to applications such as hand sanitizers, because the initial value of concentration of peroxynitrous acid (HOONO) is relatively great while the way of a change in concentration of peroxynitrous acid (HOONO) with passage of time in the case of concentration of sodium nitrite in the water solution of 2000 micro-molar is appropriate to applications such as spraying in a room, because concentration of peroxynitrous acid (HOONO) is kept at a significant value for a relatively long time.

In the embodiments described above, sodium nitrite was added to the liquid from which mist is generated in order to increase concentration of peroxynitrous acid (HOONO). In general, concentration of peroxynitrous acid (HOONO) can be increased by adding nitrous acid or nitrite ion to the liquid from which mist is generated.

An atomizer according to an embodiment is provided with a mist generator and a plasma producer. The plasma producer is provided with a tube of dielectric material, an external electrode installed on an outside surface of the tube of dielectric material, an internal electrode installed on an inside surface of the tube of dielectric material and a high-frequency power supply for applying a high-frequency voltage between the external electrode and the internal electrode. The plasma producer is configured such that mist that is sent from the mist generator to the tube of dielectric material is plasma-activated while passing through the tube of dielectric material and emitted from an end of the tube of dielectric material. The external electrode and the internal electrode are located such that location of the external electrode and location of the internal electrode does not overlap or merely partially overlap in the longitudinal direction of the tube of dielectric material.

The plasma producer according to the present embodiment can reduce an amount of mist that adheres to the inside surface of the tube of dielectric material and therefore can emit a greater amount of plasma-activated mist than a plasma producer of a conventional structure. Further, the plasma producer according to the present embodiment can generate active species required to generate peroxynitrous acid (HOONO) such that each ratio between the generated active species is in a range that is desirable for the generation of peroxynitrous acid (HOONO) and therefore can make a generation rate of peroxynitrous acid (HOONO) greater than in the case of a plasma producer of a conventional structure. Peroxynitrous acid (HOONO) has various effects such as sterilizing one, virucidal one and deodorant one.

In an atomizer according to another embodiment, the external electrode and the internal electrode are located such that location of the external electrode and location of the internal electrode merely partially overlap in the longitudinal direction of the tube of dielectric material.

In the atomizer according to the present embodiment, electric discharge between both electrodes can easily occur, because location of the external electrode and location of the internal electrode merely partially overlap with each other in the longitudinal direction of the tube of dielectric material.

In an atomizer according to another embodiment, a range of frequency of the high-frequency power supply is from 1 kilohertz to 100 kilohertz and a range of peak-to-peak value of the voltage is from 1 kilovolt to 30 kilovolts.

In an atomizer according to another embodiment, the mist generator is configured so as to form droplets of diameter in a range from 0.5 micrometers to 30 micrometers.

In an atomizer according to another embodiment, a diameter of the tube of dielectric material is in a range from 0.5 millimeters to 20 millimeters.

In an atomizer according to another embodiment, the internal electrode is a tube inside the tube of dielectric material.

In an atomizing method according to an embodiment, plasma-activated mist is generated form a liquid consisting mainly of water using one of the atomizers described above is used.

By the atomizing method according to the present embodiment, an amount of mist that adheres to the inside surface of the tube of dielectric material can be reduced and therefore an amount of emitted plasma-activated mist can be made greater than in the case of a conventional method using a plasma producer of a conventional structure. Further, by the atomizing method according to the present embodiment, the plasma producer can generate active species required to generate peroxynitrous acid (HOONO) such that each ratio between the generated active species is in a range that is desirable for the generation of peroxynitrous acid (HOONO) and therefore a generation rate of peroxynitrous acid (HOONO) can be made greater than in the case of a conventional method using a plasma producer of a conventional structure. Peroxynitrous acid (HOONO) has various effects such as sterilizing one, virucidal one and deodorant one.

In an atomizing method according to another embodiment, the liquid used for generating mist is water.

In an atomizing method according to another embodiment, the liquid used for generating mist is an aqueous solution containing nitrous acid or nitrite ion.

In the atomizing method according to the present embodiment, a generation rate of peroxynitrous acid (HOONO) having sterilizing effects can be made greater than in the case of the liquid consisting of water.

In an atomizing method according to another embodiment, the liquid contains sodium nitrite and a range of molar concentration of sodium nitrite is from 1 micro-molar to 100 milli-molar.

What is claimed is:

1. An atomizing method, comprising:
providing an atomizer including a mist generator and a plasma producer, wherein the plasma producer is provided with a tube of dielectric material, an external electrode installed on an outside surface of the tube of dielectric material, an internal electrode installed on an inside surface of the tube of dielectric material and a high-frequency power supply for applying a high frequency voltage between the external electrode and the internal electrode and is configured such that mist that is sent from the mist generator to the tube of dielectric material is plasma-activated while passing through the tube of dielectric material and emitted from an end of the tube of dielectric material, and wherein
the external electrode and the internal electrode are located such that location of the external electrode and location of the internal electrode does not overlap or merely partially overlap in the longitudinal direction of the tube of dielectric material, the method further comprising
continuously generating peroxynitrous acid by generating hydrogen peroxide and nitrous acid using plasma-activated water mist, using said atomizer.

2. The atomizing method according to claim 1, wherein the external electrode and the internal electrode are located such that location of the external electrode and location of the internal electrode merely partially overlap in the longitudinal direction of the tube of dielectric material.

3. The atomizing method according to claim 1, wherein a range of frequency of the high-frequency power supply is from 1 kilohertz to 100 kilohertz and a range of peak-to-peak value of the voltage is from 1 kilovolt to 30 kilovolts.

4. The atomizing method according to claim 1, wherein the mist generator is configured so as to form droplets of diameter in a range from 0.5 micrometers to 30 micrometers.

5. The atomizing method according to claim 1, wherein a diameter of the tube of dielectric material is in a range from 0.5 millimeters to 20 millimeters.

6. The atomizing method according to claim 1, wherein the internal electrode is a tube inside the tube of dielectric material.
